(19) United States
(12) Patent Application Publication
(10) Pub. No.: US 2025/0164424 A1
(43) Pub. Date: May 22, 2025

(54) Method for evaluating suitability of a pharmaceutical container for lipid-based carrier systems

(71) Applicant: SCHOTT Pharma AG & Co. KGaA, Mainz (DE)

(72) Inventors: Matthias BICKER, Mainz (DE); Christoph BRÜNING, Kiedrich (DE); Anne SPENDE, Wiesbaden (DE); Flor TOLEDO RODRÍGUEZ, Frankfurt am Main (DE); Michaela GESTERKAMP; Uwe ROTHHAAR, Birkenheide (DE)

(21) Appl. No.: 18/840,987

(22) PCT Filed: Mar. 25, 2022
(86) PCT No.: PCT/EP2022/058035

§ 371 Date: Aug. 23, 2024

(51) Int. Cl. G01N 23/2258; A61J 1/14

(52) U.S. Cl. CPC G01N 23/2258; A61J 1/1468

(57) ABSTRACT

A method for evaluating suitability of a pharmaceutical container for lipid-based carrier systems. Lipid-based carrier systems are sensitive pharmaceutical vehicles which require a pharmaceutical container that meets the necessary standards for storage and shipment.

The invention relates to a method for evaluating suitability of a pharmaceutical container for lipid-based carrier systems. Lipid-based carrier systems are sensitive pharmaceutical vehicles which require a pharmaceutical container that meets the desired standards for storage and shipment.

### BACKGROUND

Lipid-based carrier systems, such as e.g. lipid nanoparticles (LNPs) are a modern drug delivery vehicle that is used for pharmaceutically active, sensitive ingredients, for example mRNA.

LNPs used for mRNA vaccines against SarS-COV-2 are based on four chemically different types of lipids, i.e. phospholipids, cholesterol, PEG-modified lipids and cationic lipids. Cationic lipids bind mRNA due to their opposite molecular charges. mRNA vaccines are chemically sensitive and require high demands on their storage conditions, for example temperatures well below −20° C. to preserve the drug.

Accordingly, high demands are placed on the containers for the storage and transport of mRNA vaccines. One particular problem in the context of mRNA vaccines is that adhesion and possible inactivation of the supramolecular structures to the container wall has been observed. In that context, there remains a challenge to assess pharmaceutical containers for their suitability for lipid-based carrier systems with respect to storage and shipment.

A wealth of pharmaceutical containers is on the market, most of which are glass-based, while some are polymer-based. Pharmaceutical containers may also be coated to alter their surface properties, in particular with respect to adhesion properties.

Yet, a method for evaluating the suitability of a pharmaceutical container for lipid-based carrier systems remains elusive.

### SUMMARY OF THE INVENTION

In a first aspect, the invention relates to a method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the selected signals from the ToF-SIMS data, obtaining one or more factors;
obtaining corresponding scores for the one or more factors from the MCR analysis; and
evaluating at least one score of the one or more factors by comparison to a reference score.

The method according to the invention advantageously allows to collect relevant information in the form of ToF-SIMS data from the inner surface of the container which has previously undergone a typical treatment, i.e. incubation the container with a lipid-based carrier system. The ToF-SIMS data provide a wealth of information on relevant molecular (ion) species that are found in the coating and/or on the container. The method advantageously allows to reduce this complex set of information via MCR (Multivariate Curve Resolution) analysis on the ToF-SIMS data using one or more factors that are representative of relevant compound classes found in the coating and/or on the container. The method provides a single score value for each chosen factor that can be easily compared to reference conditions.

The evaluation of the score of one or more factors by comparison to a reference pharmaceutical container or reference conditions makes this method versatile, i.e. an experimental frame of reference may be chosen which suits the pharmaceutical container, e.g. a coated container may be compared over an uncoated container. Further, the method allows free choice of mathematical evaluation, e.g. by assessing difference or quotient values. Also, threshold values may differ for certain applications and requirements set by the authorities.

In a related aspect, the invention relates to a method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the ToF-SIMS data obtaining one or more factors;
selecting one or more factors, wherein the one or more factors include lipid-factor1;
obtaining a corresponding score for lipid factor 1 from the MCR analysis;
dividing the score of lipid factor 1 by the analogous score of a reference pharmaceutical container to obtain a quotient for lipid-factor1; and
assessing suitability of the pharmaceutical container for lipid-based carrier systems based on the criterion whether the quotient for lipid-factor1 is less than 0.67, or less than 0.5.

In a related aspect, the invention relates to a method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the ToF-SIMS data obtaining one or more factors;
selecting one or more factors, wherein the one or more factors include silicon-organic factor1;
obtaining a corresponding score for silicon-organic factor1 from the MCR analysis;
dividing the score of silicon-organic factor1by the analogous score of a reference pharmaceutical container to obtain a quotient for silicon-organic factor1; and
assessing suitability of the pharmaceutical container for lipid-based carrier systems based on the criterion whether the quotient for silicon-organic factor1 is more than 2.0.

In a related aspect, the invention relates to a method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the ToF-SIMS data obtaining one or more factors;
obtaining a corresponding score for organic factor1 from the MCR analysis;
dividing the score of organic factor1 by the analogous score of a reference pharmaceutical container to obtain a quotient for organic factor1; and
assessing suitability of the pharmaceutical container for lipid-based carrier systems based on the criterion whether the quotient for organic factor1 is more than 0.2.

### DETAILED DESCRIPTION

In a first aspect, the invention relates to a method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the selected signals from the ToF-SIMS data, obtaining one or more factors;
obtaining corresponding scores for the one or more factors from the MCR analysis; and
evaluating at least one score of the one or more factors by comparison to a reference score.

The acquired ToF-SIMS data uncover a multitude of molecular (ion) species signals which originate from the container and/or the coating, e.g. the glass or a polymer substrate, siloxanes, phospolipids, other lipids, buffer ingredients, as well as unspecific organic molecules and inorganic salts. The high-dimensional data space created by the secondary ions, which are treated as individual variables, are then replaced by new variables, the so-called factors. The position of the factors within the high-dimensional data space is described by loadings. The application of MCR (Multivariate Curve Resolution) analysis on the loadings with respect to one or more of the (specific) factors yields a score.

The skilled person knows that ToF-SIMS provides masses of molecular fragments which are detected by the instrument. Subsequently, using analysis tools and/or appropriate libraries, the obtained masses are assigned to, or interpreted as, specific molecular (ion) species. FIG. 5A and FIG. 5B show the lists of relevant molecular fragments which may be assigned to specific masses obtained with the ToF-SIMS method when applied to the pharmaceutical containers of the invention.

In one embodiment, the method comprises selecting between 10 to 1000 signals, or between 20 to 800 signals, or between 50 to 500 signals, or between 100 to 200 signals, from the ToF-SIMS data. The skilled person knows how to choose relevant molecular (ion) species from ToF-SIMS data depending on the investigated container. In one embodiment, the method comprises selecting 10 or more signals, or 20 or more signals, or 50 or more signals, or 100 or more signals. In one embodiment, the method comprises selecting 1000 signals or less, 800 signals or less, 500 signals or less, or 200 signals or less.

In one embodiment, the method comprises selecting one or more factors, e.g. 3 to 8 factors, three factors, four factors, five factors, or three to five factors, wherein the factors are selected from lipid factor1, silicon-organic factor1, silicon-inorganic factor1 and organic factor1.

In one embodiment, the pharmaceutical container is a syringe, a cartridge, an ampoule or a glass vial, wherein the pharmaceutical container is a glass container or a polymer container.

In one embodiment, the step incubating the container with a lipid-based carrier system comprises the following steps:
filling the container with a reference composition, containing a lipid-based carrier system,
storing the filled container,
emptying, cleaning and drying the container.

In one embodiment of the method, the filled container is stored for a period of at least 3 hours, or at least 6 hours;
the filled container is stored at a temperature below 10° C. and/or
the filled container is stored at a temperature below −10° C., or even below −50° C.

In one embodiment of the method, before filling the container with the reference composition, the container is cleaned, preferably with water.

In one embodiment of the method, the reference composition
comprises a lipid-based carrier system, such as LNPs, and water, and optionally a buffer; or
comprises or is the Comirnaty® vaccine drug product (license number EU/1/20/1528).

In one embodiment, the step incubating the container with a lipid-based carrier system comprises the following steps:
filling the container with a reference LNP-composition, freezing to −80° C., incubating for 12 hours at −80° C., and then thawing to 5° C. within 12 hours; or
cleaning the container with UltraPure water (purity 1 analogue DIN ISO 3696 with ≤0.1 μS/cm at 25° C.), drying under laminar-flow conditions, filling the container with a reference LNP-composition, freezing to −80° C., incubating for 12 hours at −80° C., and then thawing to 5° C. within 12 hours,
   wherein the reference LNP-composition is the Comirnaty vaccine (license number EU/1/20/1528), or
   wherein the reference LNP-composition contains 7.2 mg/ml (4-hydroxybutyl)azanediyl)bis(hexane-6,1-diyl)bis(2-hexyldecanoate), 0.83 mg/mL 2 [(polyethylene glycol)-2000]-N, N-ditetradecylacetamide, 1.5 mg/mL 1,2-distearoyl-sn-glycero-3-phosphocholine, and 3.3 mg/mL cholesterol, in phosphate-buffered saline (PBS) (pH 7.4) with a saccharide content of 10 wt. % and the following concentrations:

| Compound | Concentration | Concentration |
|---|---|---|
| (salt or sugar) | (mmol/L) | (g/L) |
| NaCl | 137 | 8.0 |
| KCl | 2.7 | 0.2 |
| Na₂HPO₄ | 10 | 1.42 |
| KH₂PO₄ | 1.8 | 0.24 |
| Sucrose | | 100 |

The incubation step with a lipid-based carrier system may differ for a glass container vis-à-vis a polymer container and is performed under well-defined conditions to establish reproducibility and comparison between the container and a reference container.

In one embodiment, the reference LNP-composition is the Comirnaty vaccine (license number EU/1/20/1528), or

In one embodiment, the reference LNP-composition contains 7.2 mg/ml (4-hydroxybutyl)azanediyl)bis(hexane-6,1-diyl)bis(2-hexyldecanoate), 0.83 mg/mL 2 [(polyethylene glycol)-2000]-N, N-ditetradecylacetamide, 1.5 mg/mL 1,2-distearoyl-sn-glycero-3-phosphocholine, and 3.3 mg/mL cholesterol, in phosphate-buffered saline (PBS) (pH 7.4) with a saccharide content of 10 wt. % and the following concentrations:

| Compound | Concentration | Concentration |
|---|---|---|
| (salt or sugar) | (mmol/L) | (g/L) |
| NaCl | 137 | 8.0 |
| KCl | 2.7 | 0.2 |
| Na₂HPO₄ | 10 | 1.42 |
| KH₂PO₄ | 1.8 | 0.24 |
| Sucrose | | 100 |

In one embodiment, the one or more factors further include silicon-organic factor1, silicon-inorganic factor1 and organic factor1.

In one embodiment, the step performing MCR analysis on the ToF-SIMS data, wherein each factor has a factor-specific MCR loading which indicates a conceptional component in an n-dimensional compositional space which can be attributed to each of the one or more factors, wherein the factor-specific MCR loading characterizes the one or more factors by listing the ions/masses that contribute to the definition of said factor.

The one or more factors may be selected from lipid factor1, silicon-organic factor1, silicon-inorganic factor1 and organic factor1, wherein each of the factors has a factor-specific MCR loading which indicates a conceptional component in said n-dimensional compositional space which can be attributed to said one or more factors, wherein the factor-specific MCR loading characterizes the one or more factors by listing the ions that contribute to the definition of said factor. The conceptional component represents compound classes, such as e.g. lipids, siloxanes, and glass-typical silicon species. As such the conceptional component is not present in the coating or on the container.

In one embodiment, the lipid factor1 includes, in its factor specific MCR loading, one or more of the following ions: fatty acid-ions; [CₙH₂ₙ₋₁O₂]⁻, wherein n is 10, 12, 14, 16 or 18; [CₙH₂ₙ₋₃O₂]⁻, wherein n is 10, 12, 14, 16 or 18; [CₙH₂ₙ₋₅O₂]⁻, wherein n is 16 or 18; phosphatidyl-choline ions; [(CH)ₙH₂O₄P]⁻, wherein n=0, 1, 2 or 3.

In one embodiment, the silicon-organic factor1 includes, in its factor specific MCR loading, one or more of the following ions: silane species, silicon-carbon species, polysiloxane species based on the formula [OSiR₁R₂]ₙ⁻, wherein R₁ and R₂ are independently selected from methyl, ethyl, propyl, and wherein n is any integer between 2 and 10.

In one embodiment, the silicon-inorganic factor1 includes, in its factor specific MCR loading, one or more of the following ions: silicon species; aluminium-species and/or boron species; halogen species.

In one embodiment, the lipid factor1 includes, in its factor specific MCR loading, one or more of the following ions: [C₁₀H₁₇O₂]⁻, [C₁₀H₁₉O₂]⁻, [C₁₂H₂₁O₂]⁻, [C₁₆H₂₉O₂]⁻, [C₁₆H₃₁O₂]⁻, [C₁₆H₃₂O₂]⁻, [C₁₈H₃₁O₂]⁻, [C₁₈H₃₃O₂]⁻, [C₁₈H₃₅O₂]⁻, [PO₃]⁻, [PH₂O₄]⁻, [CH₃O₄P]⁻, [C₂H₄O₄P]⁻.

In one embodiment, the silicon-organic factor1 includes, in its factor specific MCR loading, one or more of the following ions: [Sic]⁻, [SiCH₃O]⁻, [SiCH₃O₂]⁻, [SiC₂H₅O]⁻, [Si₂CHO₂]⁻, [SiC₃H₉O]⁻, [Si₂C₅H₁₅O₂]⁻, [Si₃C₅H₁₅O₄]⁻.

In one embodiment, the silicon-inorganic factor1 includes, in its factor specific MCR loading, one or more of the following ions: OH⁻, Al⁻, Si⁻, P⁻, Cl⁻, NaO⁻, AlO⁻, BO₂⁻, SiHO⁻, AlO₂⁻, SiO₂⁻, SiH₅O₂⁻; Si₃H₃O₂⁻, Si₂HO₅⁻.

In one embodiment, the lipid factor1 includes, in its factor specific MCR loading, at least 5 of the following ions: [C₁₀H₁₉O₂]⁻, [C₁₂H₂₁O₂]⁻, [C₁₆H₂₉O₂]⁻, [C₁₆H₃₁O₂]⁻, and [C₁₈H₃₅O₂]⁻.

In one embodiment, the silicon-organic factor1 includes, in its factor specific MCR loading, at least 5 of the following ions: [SiCH₃O], [SiCH₃O₂]⁻, [SiC₂H₅O]⁻, [SiC₃H₉O]⁻, and [Si₂C₅H₁₅O₂]⁻.

In one embodiment, the silicon-inorganic factor1 includes, in its factor specific MCR loading, at least 5 of the following ions: OH⁻, Si⁻, SiO₂⁻, SiH₅O₂⁻, and Si₃H₃O₂⁻.

Method according to any one of the preceding claims, wherein the step evaluating the score of the one or more factors by comparison to a reference score comprises one or more of the following steps
obtaining a reference score from a reference container; and/or
obtaining a reference value for said score, e.g. from literature.

The “reference container”, as used herein, may be an uncoated container. The reference container may be of the same dimensions and materials and bulk composition as the pharmaceutical container. For example, the pharmaceutical container may be coated whereas the reference container may be uncoated.

In one embodiment of the method, the step evaluating the score of the one or more factors by comparison to a reference score comprises one or more of the following steps:
comparing the score, e.g. of lipid-factor1, of the pharmaceutical container with the reference score;
assessing whether a difference of the score, e.g. of lipid-factor1, of the pharmaceutical container and the reference score, e.g. of lipid-factor1, is below or above a pre-set threshold value; and/or
assessing whether a quotient of the score, e.g. of lipid-factor1, of the pharmaceutical container and the reference score, e.g. of lipid-factor1, is below or above a pre-set threshold value.

In one embodiment of the method, the evaluation results in a positive or negative answer which, respectively, indicates fitness of suitability or lack of suitability.

In one embodiment of the method, the comparison to a reference pharmaceutical container comprises the following steps:
providing a reference container having an inner surface and an outer surface, wherein at least part of the inner surface is coated with a coating, wherein the reference container has the same dimensions and the same composition as the pharmaceutical container;
incubating the reference container with a lipid-based carrier system;
acquiring ToF-SIMS data from the inner surface of the reference container;
selecting between 10 to 1000 signals from the ToF-SIMS data;
performing MCR (Multivariate Curve Resolution) analysis on the ToF-SIMS data obtaining one or more factors;
obtaining corresponding scores for the one or more factors from the MCR analysis; and
assessing whether the difference of the score of the one or more factors of the pharmaceutical container and the score of the one or more factors of the reference pharmaceutical container is below or above a pre-set threshold value; or
assessing whether the quotient of the score of the one or more factors of the pharmaceutical container and the score of the one or more factors of the reference pharmaceutical container is below or above a pre-set threshold value.

### RELATED ASPECTS

Advantageously, the method according to the invention can be adapted to select individual factors of relevance, such as lipid factor1, silicon-organic factor1, silicon-inorganic factor1 and organic factor1. The ToF-SIMS data acquired from the inner surface of the container cover a wealth of molecular (ion) species/masses, most of which belong to at least one well-defined compound group covered by the factors. Feeding the ToF-SIMS data into the MCR (Multivariate Curve Resolution) analysis on the ToF-SIMS data using a selected factor allows direct comparison of a pharmaceutical container to a reference pharmaceutical container by means of the corresponding scores.

It is advantageous to directly compare the scores for the pharmaceutical container and the reference pharmaceutical container by a calculus-based operation and subsequent assessment of the calculated result. This calculus-based operation and subsequent assessment can be done for the individual factors, but is also accessible for combinations of several individual factors.

In a related aspect, the invention relates to a method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the ToF-SIMS data using the factor;
selecting one or more factors, wherein the one or more factors include lipid-factor1;
obtaining a corresponding score for the factor from the MCR analysis;
dividing the score of the factor by the analogous score of a reference pharmaceutical container to obtain a quotient for lipid-factor1; and
assessing suitability of the pharmaceutical container for lipid-based carrier systems based on the criterion whether the quotient for lipid-factor1 is less than 0.5.

In a related aspect, the invention relates to a method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the ToF-SIMS data using the factor;
selecting one or more factors, wherein the one or more factors include silicon-organic factor1;
obtaining a corresponding score for the factor from the MCR analysis;
dividing the score of the factor by the analogous score of a reference pharmaceutical container to obtain a quotient for silicon-organic factor1; and
assessing suitability of the pharmaceutical container for lipid-based carrier systems based on the criterion whether the quotient for silicon-organic factor1 is more than 2.0.

In a related aspect, the invention relates to a method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the ToF-SIMS data using the factor;
selecting one or more factors, wherein the one or more factors include organic factor1;
obtaining a corresponding score for the factor from the MCR analysis;
dividing the score of the factor by the analogous score of a reference pharmaceutical container to obtain a quotient for organic factor1; and
assessing suitability of the pharmaceutical container for lipid-based carrier systems based on the criterion whether the quotient for organic factor1 is more than 0.2.

In one embodiment of the method, the suitability of the pharmaceutical container is evaluated based on
assessing suitability of the pharmaceutical container for lipid-based carrier systems based on the criterion whether the quotient for lipid-factor1 is less than 0.5; and
assessing suitability of the pharmaceutical container for lipid-based carrier systems based on the criterion whether the quotient for silicon-organic factor1 is more than 2.0.

Pharmaceutical containers with a combined low quotient for lipid-factor1 and an elevated quotient for silicon-organic factor1 may be considered as very suitable for lipid-based carrier systems, because the results indicate that there is only little adsorption of the lipid-based carrier and its individual constituting components, while a previously provided coating of the pharmaceutical container remains intact. An intact coating of the pharmaceutical may be evidenced by silicon-organic species, such as siloxane, which are intended to protect the lipid-based carrier from adsorption to the glass.

In one embodiment of the method, the suitability of the pharmaceutical container is evaluated based on
assessing suitability of the pharmaceutical container for lipid-based carrier systems based on the criterion whether the quotient for lipid-factor1 is less than 0.5; and
assessing suitability of the pharmaceutical container for lipid-based carrier systems based on the criterion whether the quotient for organic factor1 is more than 0.2.

Analogously, pharmaceutical containers with a combined low quotient for lipid-factor1 and an elevated quotient for organic factor1 may be considered as very suitable for lipid-based carrier systems, because the results indicate that there is only little adsorption of the lipid-based carrier and its individual constituting components, while the pharmaceutical polymer container remains intact. An intact polymer container may be evidenced by organic species which evolve from the polymer itself.

### Container

The following disclosure relates to the pharmaceutical container and/or the reference container.

In one embodiment, the container is a glass container or a polymer container.

In one embodiment, the container comprises a cyclic olefin copolymer. In one embodiment, the container comprises a cyclic olefin polymer.

In one embodiment, the container has one or more of the following properties:
a wall thickness between 0.50 and 10.0 mm, or between 1.00 and 4.00 mm; and/or
a volume capacity of the container of 0.1 ml to 1000 ml, 0.5 ml to 500 ml, 1 ml to 250 ml, 2 ml to 30 ml, 2 ml to 15 ml, or about 1 ml, 2 ml, 3 ml, 4, ml, 5 ml, 6 ml, 7 ml, 8 ml, 9 ml, 10 ml, 11 ml, 12 ml, 13 ml, 14 ml or 15 ml; optionally from 5 to 15 ml.

In one embodiment, the container has a wall thickness of 0.50 mm or more, 1.00 mm or more, or 2.0 mm or more. In one embodiment, the container has a wall thickness of 10.0 mm or less, or 7.00 mm or less, or 4.0 mm or less.

In one embodiment, the container is a syringe, a cartridge, an ampoule or a vial.

### Glass Composition

The following disclosure relates to the pharmaceutical container and/or the reference container.

In one embodiment, the container comprises a glass composition comprising 50 to 90 wt. % SiO₂, and 3 to 25 wt. % B₂O₃.

In one embodiment, the container comprises a glass composition comprising aluminosilicate, optionally comprising 55 to 75 wt. % SiO₂, and 11.0 to 25.0 wt. % Al₂O₃.

In one embodiment, the container comprises a glass composition comprising 70 to 81 wt. % SiO₂, 1 to 10 wt. % Al₂O₃, 6 to 14 wt. B₂O₃, 3 to 10 wt. % Na₂O, 0 to 3 wt. % K₂O, 0 to 1 wt. % Li₂O, 0 to 3 wt. % MgO, 0 to 3 wt. % CaO, and 0 to 5 wt. % BaO.

In one embodiment, the container comprises a glass composition comprising 72 to 82 wt. % SiO₂, 5 to 8 wt. % Al₂O₃, 3 to 6 wt. B₂O₃, 2 to 6 wt. % Na₂O, 3 to 9 wt. % K₂O, 0 to 1 wt. % Li₂O, 0 to 1 wt. % MgO, and 0 to 1 wt. % CaO.

In one embodiment, the container comprises a glass composition comprising 60 to 78 wt. % SiO₂, 7 to 15 wt. B₂O₃, 0 to 4 wt. % Na₂O, 3 to 12 wt. % K₂O, 0 to 2 wt. % Li₂O, 0 to 2 wt. % MgO, 0 to 2 wt. % CaO, 0 to 3 wt. % BaO, and 4 to 9 wt. % ZrO₂.

In one embodiment, the container comprises a glass composition comprising 50 to 70 wt. % SiO₂, 10 to 26 wt. % Al₂O₃, 1 to 14 wt. B₂O₃, 0 to 15 wt. % MgO, 2 to 12 wt. % CaO, 0 to 10 wt. % BaO, 0 to 2 wt. % SrO, 0 to 8 wt. % ZnO, and 0 to 2 wt. % ZrO₂.

In one embodiment, the container comprises a glass composition comprising 55 to 70 wt. % SiO₂, 11 to 25 wt. % Al₂O₃, 0 to 10 wt. % MgO, 1 to 20 wt. % CaO, 0 to 10 wt. % BaO, 0 to 8.5 wt. % SrO, 0 to 5 wt. % ZnO, 0 to 5 wt. % ZrO₂, and 0 to 5 wt. % TiO₂.

In one embodiment, the container comprises a glass composition comprising 65 to 72 wt. % SiO₂, 11 to 17 wt. % Al₂O₃, 0.1 to 8 wt. % Na₂O, 0 to 8 wt. % K₂O, 3 to 8 wt. % MgO, 4 to 12 wt. % CaO, and 0 to 10 wt. % ZnO.

In one embodiment, the container comprises a glass composition comprising 64 to 78 wt. % SiO₂, 4 to 14 wt. % Al₂O₃, 0 to 4 wt. % B₂O₃, 6 to 14 wt. % Na₂O, 0 to 3 wt. % K₂O, 0 to 10 wt. % MgO, 0 to 15 wt. % CaO, 0 to 2 wt. % ZrO₂, and 0 to 2 wt. % TiO₂.

### Further Embodiments

In one embodiment, the method comprises the steps:
providing a pharmaceutical container; and
applying the results of the method for evaluating suitability of a pharmaceutical container for lipid-based carrier systems.

In one embodiment, the method comprises the steps:
providing a pharmaceutical container and a reference container; and
applying the results of the method for evaluating suitability of a pharmaceutical container for lipid-based carrier systems.

In one embodiment, the method comprises the steps:
providing a pharmaceutical container and a reference container; and
applying the results of the method for evaluating suitability of a pharmaceutical container for lipid-based carrier systems to determine the suitability of the pharmaceutical container for lipid-based carrier systems.

In one embodiment, the method comprises the steps:
providing a pharmaceutical container and a reference container; and
using the results of the method for evaluating suitability of a pharmaceutical container for lipid-based carrier systems to determine the suitability of the pharmaceutical container for lipid-based carrier systems.

An embodiment of this disclosure relates to a method comprising the following step(s):
using the results of the evaluation obtainable by the method according to this disclosure to evaluate the suitability of a coated container for the storage of a pharmaceutical product, preferably a lipid-based carrier system; and/or
using the results of the evaluation obtainable by the method according to this disclosure to evaluate the adhesion of a lipid-based carrier system, or parts thereof, to a coated container, preferably the coated pharmaceutical container; and/or
connecting and/or linking the results of the evaluation obtainable by the method according to this disclosure to a coated container, preferably a coated pharmaceutical container.

A further method of this disclosure includes the following steps:
producing a first and a second coated container with the same production method;
evaluating the first coated container by a method according to this disclosure, to obtain results of the evaluation of the first coated container; and
applying and/or using the results of the evaluation of the first coated container to determine the suitability of the second coated container for the storage of pharmaceutical composition, preferably in the second container, and/or using the results of the evaluation for the quality control of the first and/or second container; and/or
applying and/or using the results of the evaluation of the first coated container to determine the suitability of the second coated container for the storage of a lipid-based carrier system, preferably in the second container.

An aspect of this disclosure relates to the use of the results of the evaluation obtainable by the method according to this disclosure to evaluate the suitability of a coated container, preferably a coated pharmaceutical container, for the storage of a pharmaceutical composition, preferably a pharmaceutical composition comprising a lipid-based carrier system, and/or the quality control of the production of a coated container.

Another aspect relates to a pharmaceutical container, having attached thereto, e.g. on a label, one or more results of the method according to this disclosure, wherein optionally the result is
an indication of the suitability of the container for storing a lipid-based carrier system;
an MCR factor and/or a score;
the result of the evaluation according to one of claims 1 to 31.
the scores of lipid-factor1, silicon-organic factor1, silicon-inorganic factor1 and/or organic factor1.

This disclosure also relates to a kit, comprising:
i) a coated container, e.g. according to this disclosure, and
ii) a data sheet or storage medium comprising the results of the evaluation obtainable by the method according to this disclosure.

### LNP-Incubation

Any reference to “LNP incubated” in this disclosure means that the container or coating was incubated with LNPs before measurement. If a score is denoted as a “relative” score ratio, the respective values are to be understood as being the relative ratio of the score value of the coated container divided by the score value of an uncoated reference container based on the same MCR factor. E.g., for measuring a relative LNP-incubated MCR score ratio between a coated container and an uncoated container, wherein the uncoated container is a reference container, both containers are incubated with the same specific LNP composition as applied for the coated container. Both the coated container and the reference container are analysed via ToF-SIMS and MCR to obtain absolute MCR scores, e.g. of lipid factor1. The relative LNP-incubated MCR score ratio, e.g. of lipid factor1, is obtained by dividing the resulting MCR score of the coated container by the MCR score of the reference container. For example, the relative LNP-incubated MCR score ratio of lipid factor1 may be less than 0.5. As mentioned before, the reference container may be an uncoated container. The reference container may be of the same dimensions and materials and bulk composition as the coated container (except for the coating of course).

In an embodiment, LNP-incubation of a glass container, either being an uncoated glass container or a coated glass container, comprises cleaning the container with UltraPure water (purity 1 analogue DIN ISO 3696 with ≤0.1 μS/cm at 25° C.), drying under laminar-flow conditions, incubating the container with a reference LNP-composition by filling the container with the reference LNP-composition, freezing to −80° C., incubating for 12 hours at −80° C., and then thawing to 5° C. within 12 hours, and then emptying the containing followed by a cleaning step of the inner container surface by rinsing 10 times with ultrapure water and subsequent drying under laminar flow.

In a related embodiment, LNP-incubation of a polymer container, either being an uncoated polymer container or a coated polymer container, comprises incubating the container with a reference LNP-composition by filling the container with the reference LNP-composition, freezing to −80° C., incubating for 12 hours at −80° C., and then thawing to 5° C. within 12 hours and then emptying the containing followed by a cleaning step of the inner container surface by rinsing 10 times with ultrapure water and subsequent drying under laminar flow.

In one embodiment, the pharmaceutical container has an absolute LNP-incubated MCR score of lipid factor1 of less than 7×10¹³, less than 5×10¹³, or less than 2×10¹³. Assessing suitability of the pharmaceutical container for lipid-based carrier may be based on this criterion and the indicated score values, i.e. the container may be considered suitable, if the score is within the indicated limit.

In one embodiment, the pharmaceutical container has a relative LNP-incubated MCR score ratio of lipid factor1 of less than 0.67, less than 0.5, less than 0.3 or less than 0.13. Assessing suitability of the pharmaceutical container for lipid-based carrier may be based on this criterion and the indicated score values, i.e. the container may be considered suitable, if the score is within the indicated limit. In one embodiment, the pharmaceutical container has an absolute LNP-incubated MCR score of silicon-organic factor1 of at least 1×10¹², and/or a relative LNP-incubated MCR score ratio of silicon-organic factor1 of at least 2. Assessing suitability of the pharmaceutical container for lipid-based carrier may be based on this criterion and the indicated score values, i.e. the container may be considered suitable, if the score is within the indicated limit.

In one embodiment, the pharmaceutical container has an absolute LNP-incubated MCR score of silicon-inorganic factor1 of up to 1×10¹³, up to 5×10¹², or up to 3×10¹². Optionally, this score may be at least 0.5×10¹². Assessing suitability of the pharmaceutical container for lipid-based carrier may be based on this criterion and the indicated score values, i.e. the container may be considered suitable, if the score is within the indicated limit.

In an embodiment, the relative LNP-incubated MCR score ratio of silicon-inorganic factor1 is up to 5, up to 3, or up to 1.5. Optionally, this score ratio is at least 0.1, or at least 0.2. Assessing suitability of the pharmaceutical container for lipid-based carrier may be based on this criterion and the indicated score values, i.e. the container may be considered suitable, if the score is within the indicated limit.

In one embodiment, the pharmaceutical container has an absolute LNP-incubated MCR score of silicon-organic factor1 of at least 1×10¹², at least 2×10¹², or at least 3×10¹². Optionally, the absolute LNP-incubated MCR score of silicon-organic factor1 may reach up to 9×10¹³, up to 7×10¹³, or up to 6×10¹³. Assessing suitability of the pharmaceutical container for lipid-based carrier may be based on this criterion and the indicated score values, i.e. the container may be considered suitable, if the score is within the indicated limit.

In an embodiment, the pharmaceutical container has a relative LNP-incubated MCR score ratio of silicon-organic factor1 of at least 2, at least 3, or at least 5. Optionally, the relative LNP-incubated MCR score ratio of silicon-organic factor1 may be up to 20, up to 15, or up to 10. Assessing suitability of the pharmaceutical container for lipid-based carrier may be based on this criterion and the indicated score values, i.e. the container may be considered suitable, if the score is within the indicated limit.

In one embodiment, the pharmaceutical container has an absolute LNP-incubated MCR score of silicon-inorganic factor1 of at least 1×10¹³, and/or a relative LNP-incubated MCR score ratio of silicon-inorganic factor1 of up to 5. Assessing suitability of the pharmaceutical container for lipid-based carrier may be based on this criterion and the indicated score values, i.e. the container may be considered suitable, if the score is within the indicated limit.

In one embodiment, the container has an absolute LNP-incubated MCR score of organic factor1 of at least 1×10¹², and/or a relative LNP-incubated MCR score ratio of organic factor1 of at least 0.2. Assessing suitability of the pharmaceutical container for lipid-based carrier may be based on this criterion and the indicated score values, i.e. the container may be considered suitable, if the score is within the indicated limit.

Optionally, the container has an absolute LNP-incubated MCR score of organic factor1 of up to 9×10¹², up to 8×10¹², or up to 6×10¹². Assessing suitability of the pharmaceutical container for lipid-based carrier may be based on this criterion and the indicated score values, i.e. the container may be considered suitable, if the score is within the indicated limit.

Alternatively or in addition, the container may have corresponding, non-LNP-incubated score values. These values are obtained without LNP-incubation (“non-incubated”).

An absolute non-incubated MCR score of silicon-organic factor1 may be at least 3×10¹², at least 5×10¹², or at least 7×10¹². Optionally, the absolute non-incubated MCR score of silicon-organic factor1 may reach up to 9×10¹³, up to 7×10¹³, or up to 6×10¹³. Assessing suitability of the pharmaceutical container for lipid-based carrier may be based on this criterion and the indicated score values, i.e. the container may be considered suitable, if the score is within the indicated limit.

A relative non-incubated MCR score ratio of silicon-organic factor1 may be at least 3×10¹², at least 5×10¹², or at least 7×10¹². Optionally, the absolute LNP-incubated MCR score of silicon-organic factor1 may reach up to 9×10¹³, up to 7×10¹³, or up to 6×10¹³. Assessing suitability of the pharmaceutical container for lipid-based carrier may be based on this criterion and the indicated score values, i.e. the container may be considered suitable, if the score is within the indicated limit.

An absolute non-incubated MCR score of silicon-inorganic factor1 may be up to 1×10¹³, up to 5×10¹², or up to 3×10¹². Optionally, this score may be at least 0.5×10¹². Assessing suitability of the pharmaceutical container for lipid-based carrier may be based on this criterion and the indicated score values, i.e. the container may be considered suitable, if the score is within the indicated limit.

A relative non-incubated MCR score ratio of silicon-inorganic factor1 may be up to 5, up to 3, or up to 1.5. Optionally, this score ratio is at least 0.1, or at least 0.2. Assessing suitability of the pharmaceutical container for lipid-based carrier may be based on this criterion and the indicated score values, i.e. the container may be considered suitable, if the score is within the indicated limit.

In an embodiment, assessing suitability of the pharmaceutical container for lipid-based carrier may be based on one or more, or all, of the above criteria/score values.

### BRIEF DESCRIPTION OF THE DRAWINGS

FIG. 1 shows photographs obtained from an uncoated glass vial and a coated glass vial, wherein the glass vial was manufactured with glass tubing (Fiolax® clear, Schott AG, Germany), subjected to a reference solution and a solution containing LNPs.

FIG. 2A shows the loading for the characteristic lipid factor1 obtained from the data matrix of negative-ToF-SIMS spectra for adsorbed lipid-containing compounds on the inner surface of glass vials.

FIG. 2B shows the score values obtained for coated and uncoated glass vials from the MCR analysis based on the lipid factor1 with loading shown in FIG. 2A. Each experiment was carried out in duplicate, both on the bottom wall and on the bottom-near wall of the inner surface of the glass vials.

FIG. 3A shows the loading for the characteristic lipid factor1 obtained from the data matrix of negative-ToF-SIMS spectra for adsorbed lipid-containing compounds on the inner surface of coated and uncoated polymer syringes made of COC-polymer.

FIG. 3B shows the score values obtained for coated and uncoated polymer syringes from the MCR analysis based on the lipid factor with loading in FIG. 3A. Each experiment was carried out in duplicate, both on the bottom wall and on the bottom-near wall of the inner surface.

FIG. 4A shows the loading for the characteristic silicon-organic factor1 obtained from the data matrix of negative-ToF-SIMS spectra for silicon-organic compounds on the inner surface of coated and uncoated glass vials, glass syringes and polymer syringes.

FIG. 4B shows the score values obtained for coated and uncoated containers from the MCR analysis of the data matrix shown in FIG. 4A. Each experiment was carried out in duplicate, both on the bottom wall and on the bottom-near wall of the inner surface.

FIG. 5A shows the list of ion species which are selected from the raw ToF-SIMS data including their intensities, obtained from a glass container.

FIG. 5B shows the list of ion species which are selected from the raw ToF-SIMS data including their intensities, obtained from a polymer container.

FIG. 6A shows the loading for the characteristic silicon-inorganic factor1 obtained from the data matrix of negative-ToF-SIMS spectra for silicon-inorganic compounds on the inner surface of coated and uncoated glass vials.

FIG. 6B shows the score values obtained for coated and uncoated containers from the MCR analysis of the data matrix shown in FIG. 6A. Each experiment was carried out in duplicate, both on the bottom wall and on the bottom-near wall of the inner surface.

FIG. 7A shows the loading for the characteristic organic factor1 obtained from the data matrix of negative-ToF-SIMS spectra for organic compounds on the inner surface of coated and uncoated glass vials.

FIG. 7B shows the score values obtained for coated and uncoated glass vials from the MCR analysis of the data matrix shown in FIG. 7A. Each experiment was carried out in duplicate, both on the bottom wall and on the bottom-near wall of the inner surface.

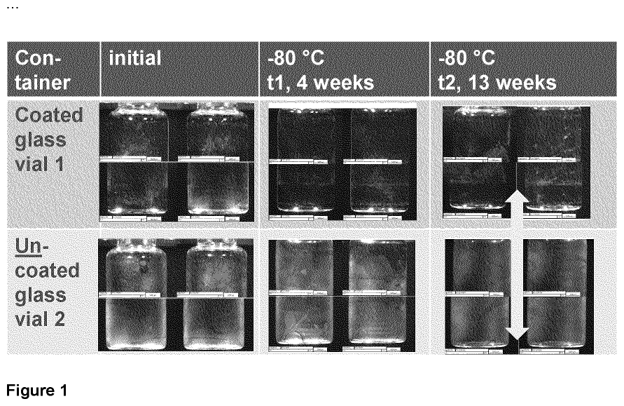

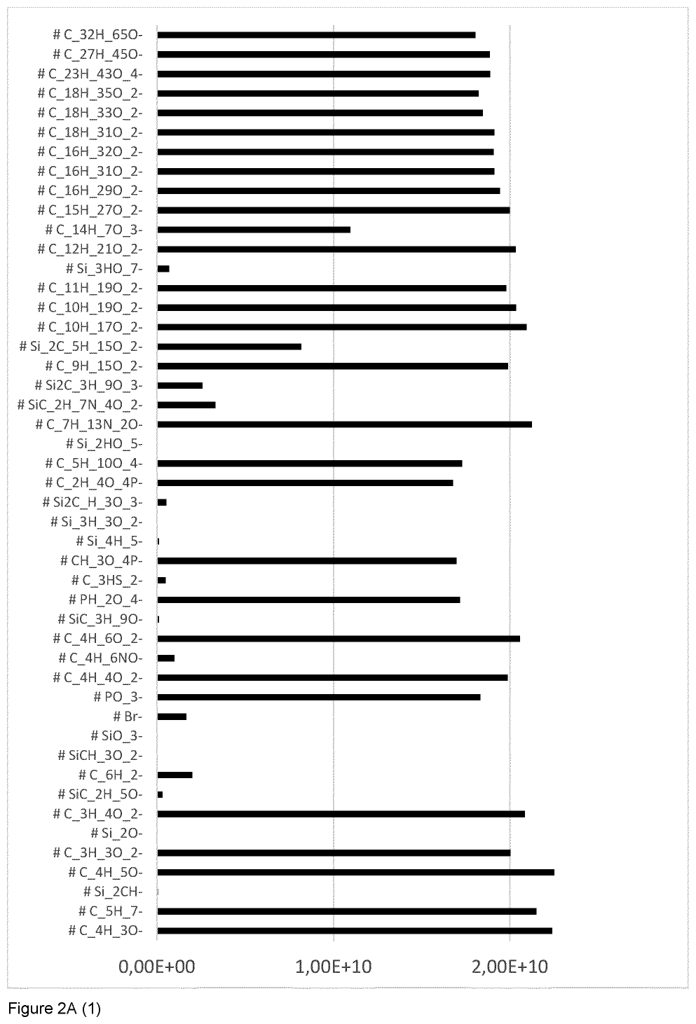

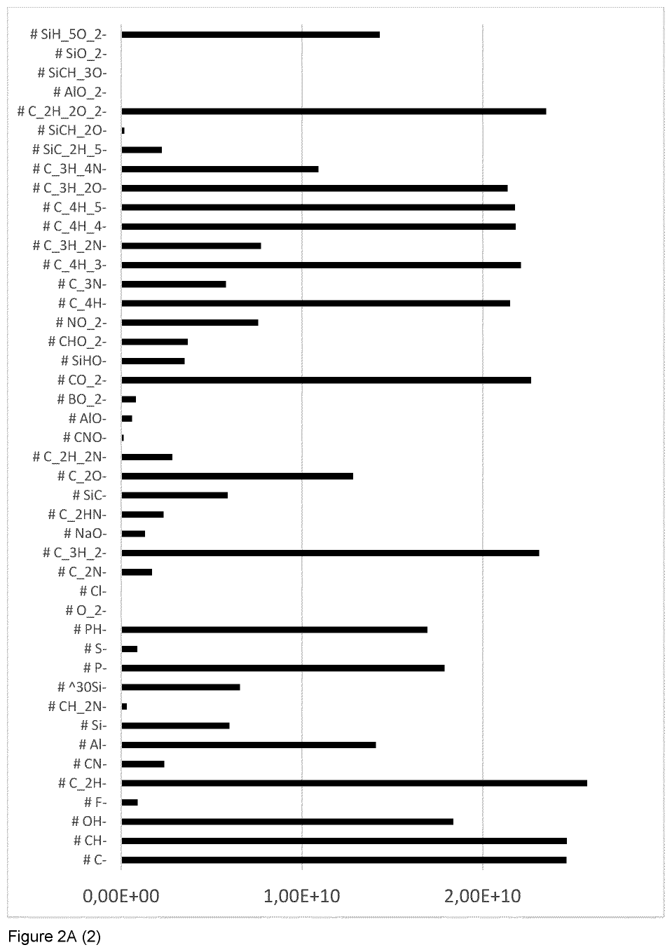

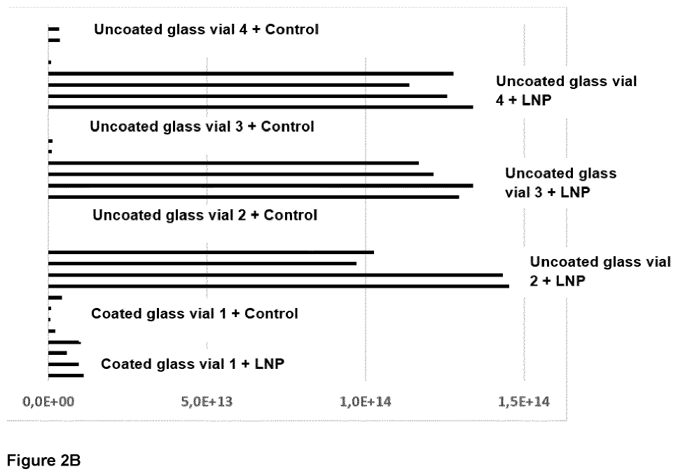

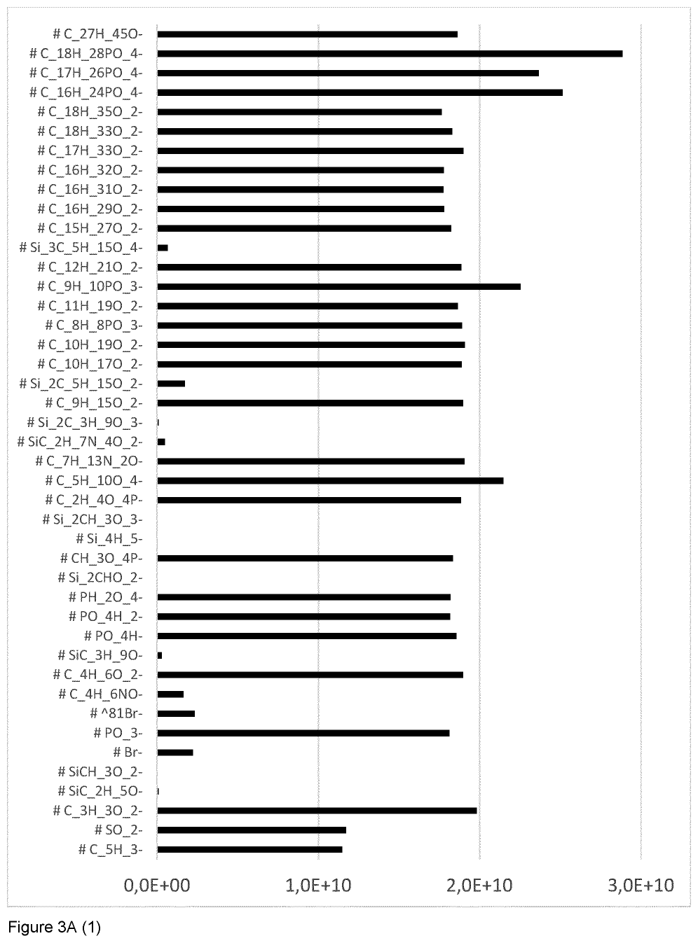

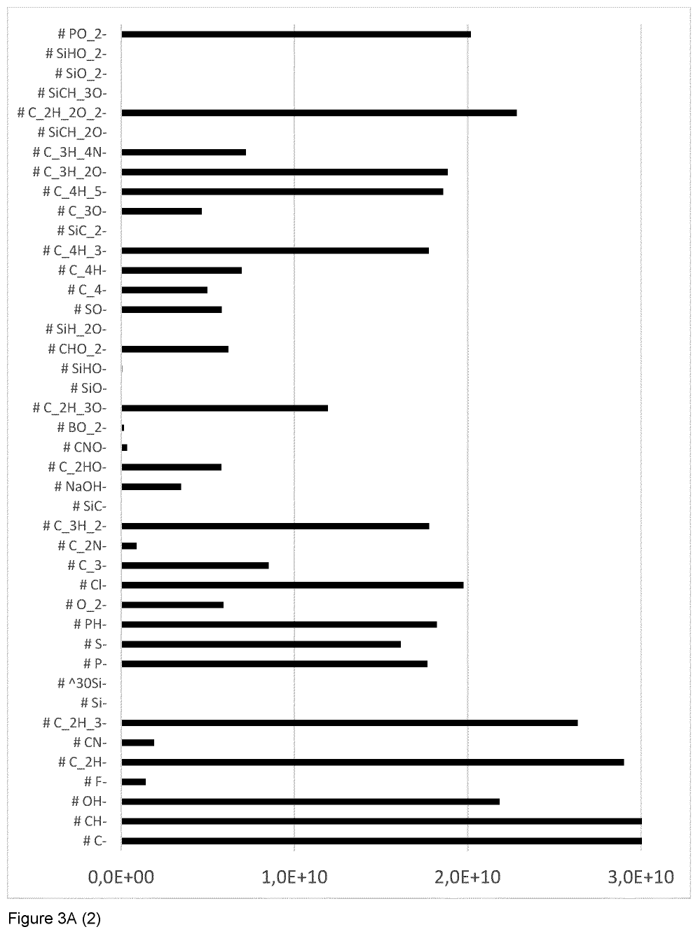

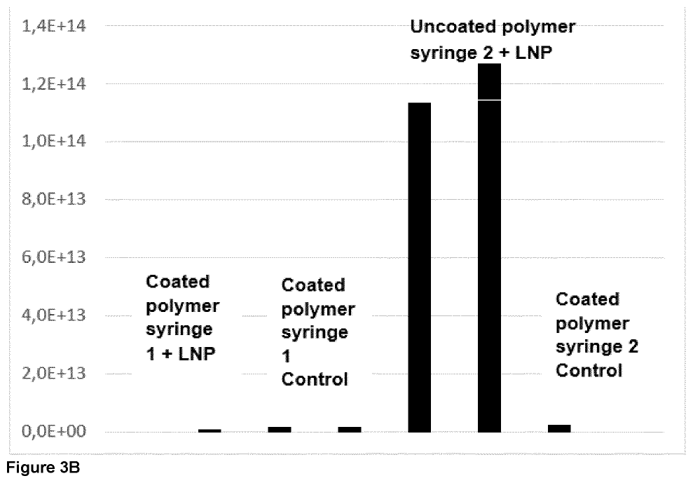

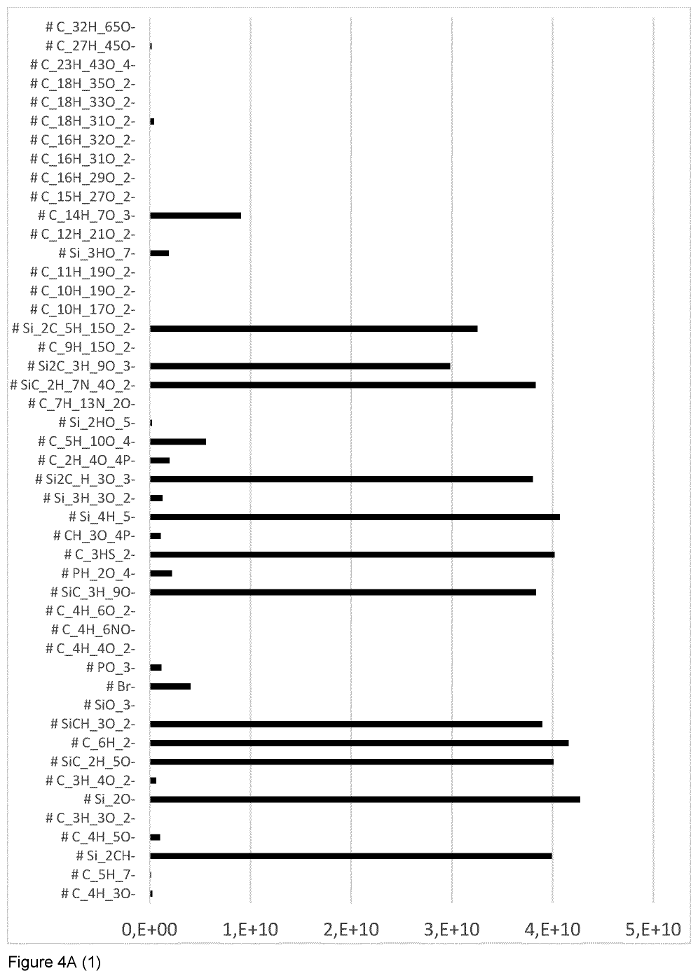

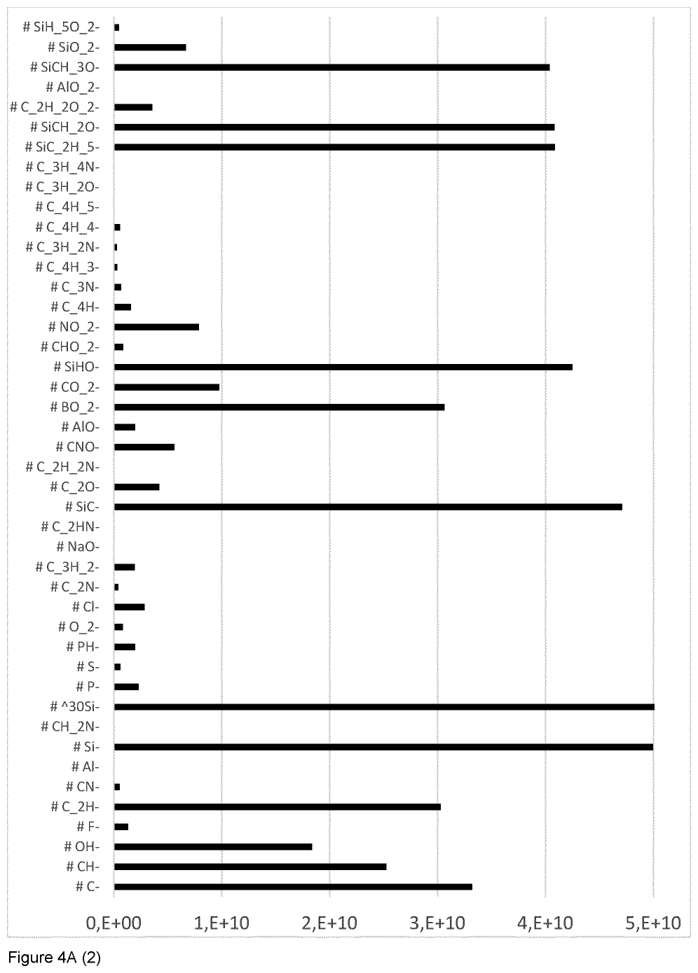

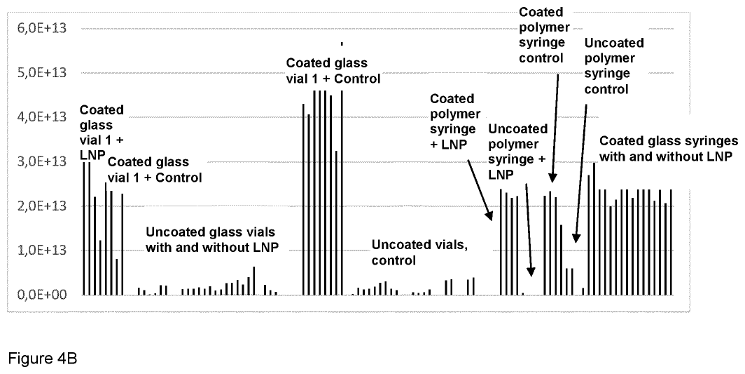

### DETAILED DESCRIPTION

### Methods and Examples

### Glass Container Preparation

A coated glass container and an uncoated glass container (the latter serving as a reference container), both having the same dimensions, same glass type and glass composition, are treated under the exact same conditions.

For measuring the LNP-incubated MCR scores, the respective container is cleaned with UltraPure water (purity 1 analogue DIN ISO 3696 with ≤0.1 μS/cm at 25° C.) and dried under laminar-flow conditions. The container is then filled with a reference LNP-composition, frozen to −80° C., incubated for 12 hours at −80° C., and then thawed to 5° C. within 12 hours.

In a first variant, the reference LNP-composition is the Comirnaty vaccine (license number EU/1/20/1528).

In a second variant, the reference-LNP contains the following lipids in the indicated amounts: 7.2 mg/mL (4-hydroxybutyl)azanediyl)bis(hexane-6,1-diyl)bis(2-hexyldecanoate), 0.83 mg/mL 2 [(polyethylene glycol)-2000]-N,N-ditetradecylacetamide, 1.5 mg/mL 1,2-distearoyl-sn-glycero-3-phosphocholine, and 3.3 mg/mL cholesterol, in phosphate-buffered saline (PBS) (pH 7.4) with a saccharide content of 10 wt. % and the following concentrations.

| Compound | Concentration | Concentration |
|---|---|---|
| (salt or sugar) | (mmol/L) | (g/L) |
| NaCl | 137 | 8.0 |
| KCl | 2.7 | 0.2 |
| Na₂HPO₄ | 10 | 1.42 |
| KH₂PO₄ | 1.8 | 0.24 |
| Sucrose | | 100 |

Both containers are analysed via ToF-SIMS and MCR analysis (see next sections).

An absolute LNP-incubated MCR score means the MCR score obtained from a single container, either the coated glass container of the uncoated (reference) glass container, for one specific factor, wherein the factor is selected from lipid factor1, silicon-organic factor1, silicon-inorganic factor1 and organic factor1, each factor having a factor-specific MCR loading.

The relative LNP-incubated MCR score ratio refers to the quotient of the MCR score of a specific factor between the coated glass container and the uncoated (reference) glass container.

### Polymer Container Preparation

A coated polymer container and an uncoated polymer container, both having the same dimensions, same glass type and glass composition, are treated under the exact same conditions.

The polymer container preparation is the same as for the glass container preparation, except that the cleaning with UltraPure water (purity 1 analogue DIN ISO 3696 with ≤0.1 μS/cm at 25° C.) and drying under laminar-flow conditions is omitted.

### ToF-SIMS (Time-of-Flight Secondary Ion Mass Spectrometry)

In the following the measuring method and the data evaluation of the specific ToF-SIMS measurement is explained in detail. For the measurement a TOF.SIMS 4 from Iontof was used. If not stated otherwise, the ToF-SIMS are measured according to ASTM E 1829 und ASTM E 2695.

### Measurement

The following parameter settings were used for the ToF-SIMS analysis:

primary ion: Ga⁺; (alternatively using a TOF.SIMS 5 and Bi⁺);

(primary ion) energy: 25000V;

measuring area: 100×100 μm²;

primary ion dose density: 6×10¹² cm⁻²;

surface discharge: via low-energetic electrons.

Spectral data were acquired for 100 s with subsequent integration.

### Sample Preparation

Each sample of a (coated) container was cut lengthwise in two pieces and positioned in such a way that the centerline of the primary ion gun of the ToF-SIMS apparatus hit the inner surface of the sample. The thus generated intrinsically ionised secondary ions have been analysed via Time-of-flight analysis and separated into different detectable mass/charge ratios. Accordingly, a high-resolution mass spectrum (Δm/m>3000 for Si) is obtained covering both atomic and molecular ion species. Die surface sensitivity covers several few monolayers.

### Data Preparation

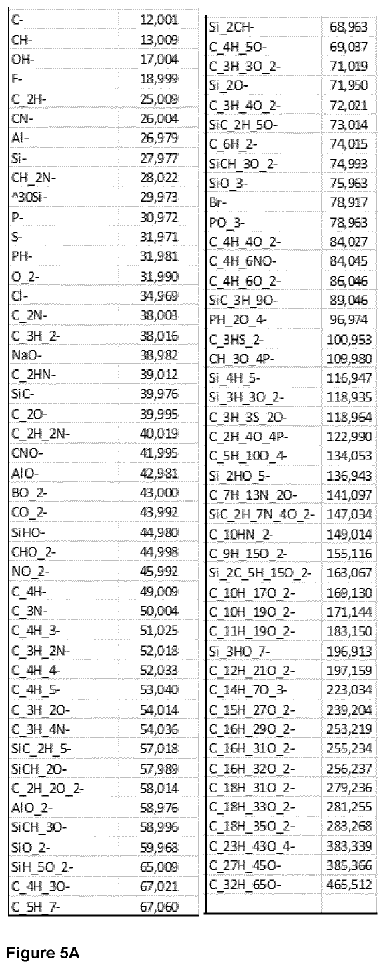

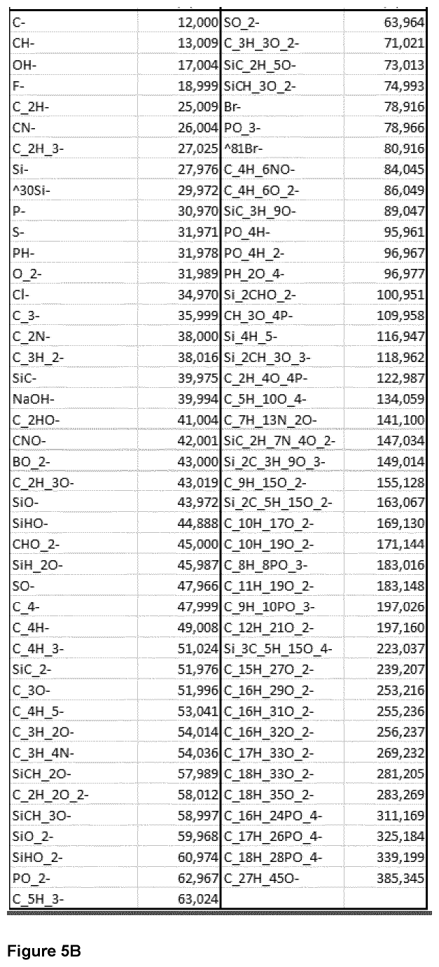

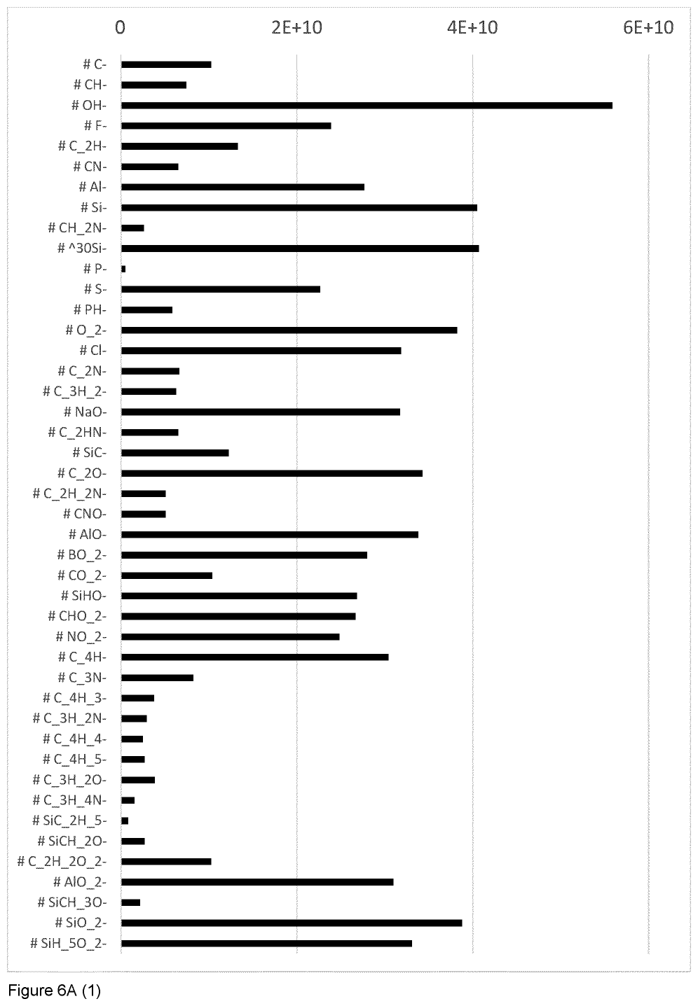

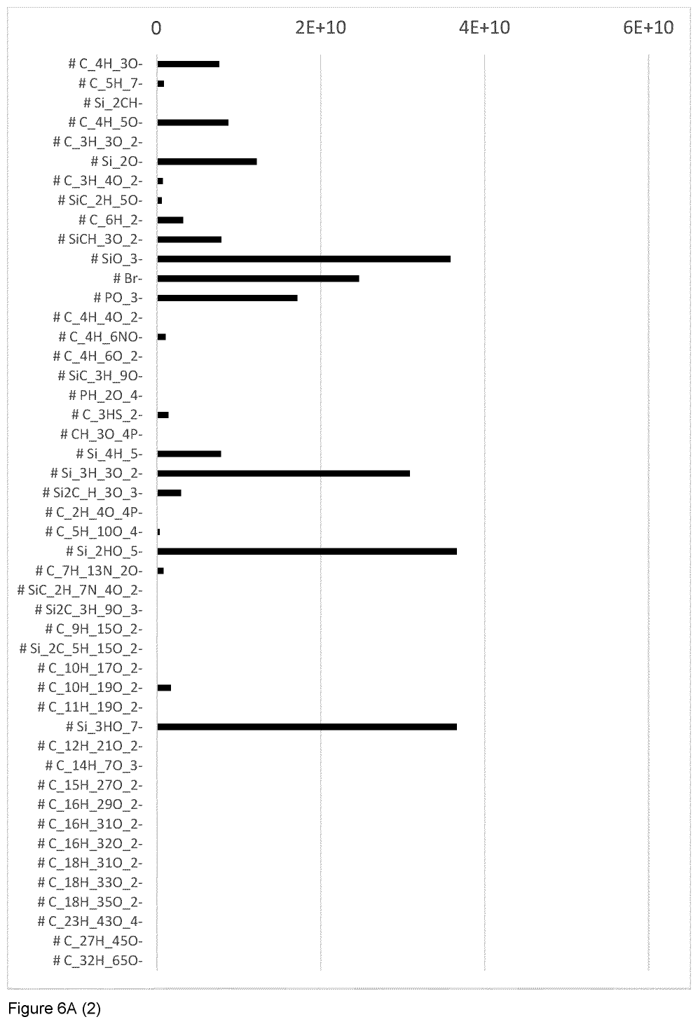

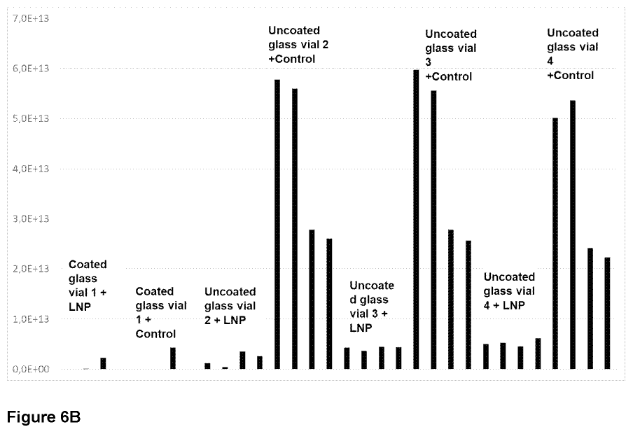

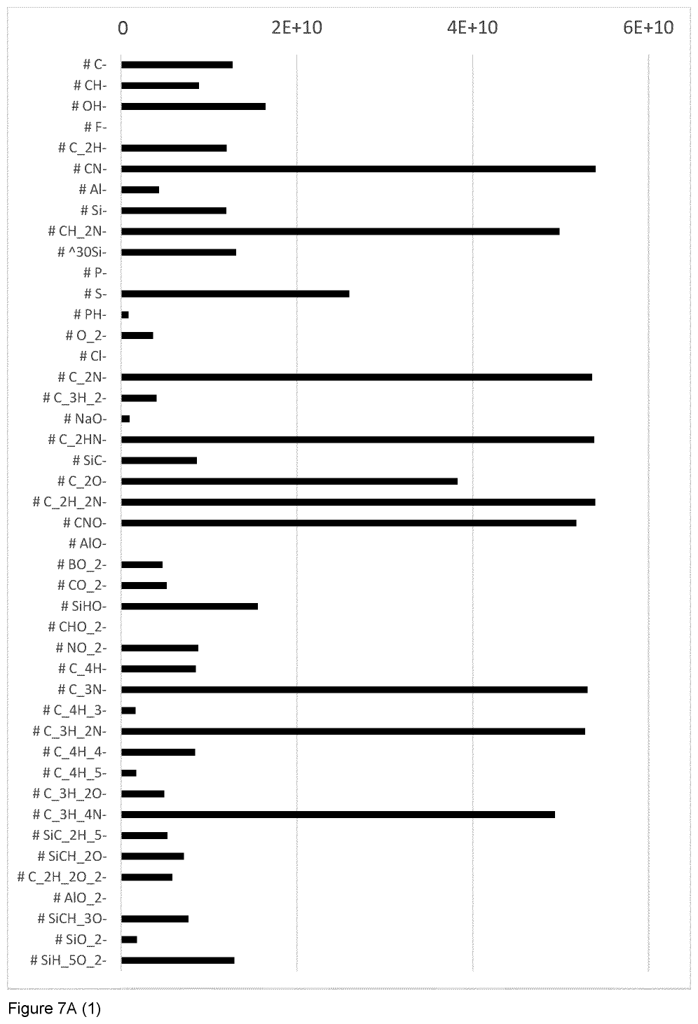

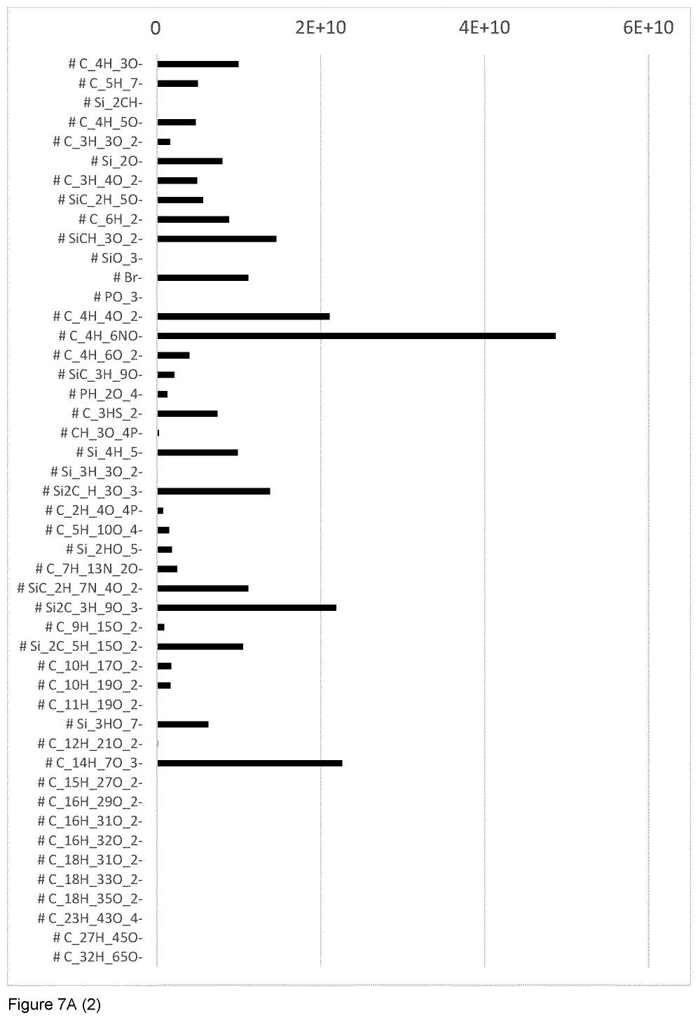

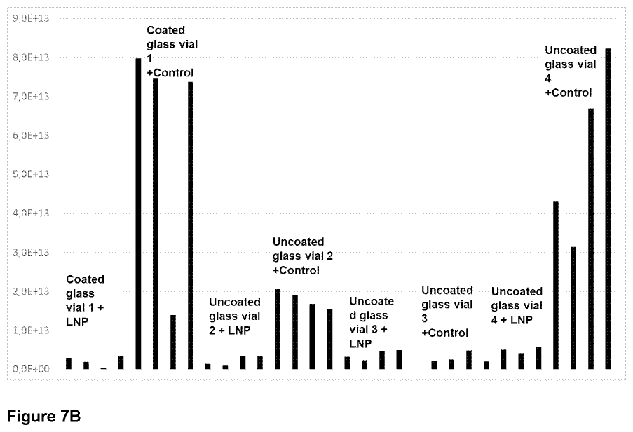

The ToF-SIMS data include n datasets consisting of ion-specific masses and their corresponding intensities. Ion species/masses are selected from the raw ToF-SIMS data, including their intensities, as indicated in FIG. 5A and FIG. 5B for, respectively, a glass container and a polymer container. The ion-specific masses relate to negative ions only because the experiment is done in negative mode. For interpretation of the masses, the spectra library from IONTOF GmbH can be used. Every ion species/mass is normalised according to its individual variance.

### Multivariate Curve Resolution (MCR)

For the identification of MCR factors, the ToF-SIMS result are subjected to a subsequent multivariate analysis via MCR (Multivariate Curve Resolution). MCR is a statistical analysis method, which in its most general approach decomposes a two-way data matrix D (m×n) into two matrices C (m×k) and ST (k×n), containing respectively pure concentration profiles and pure spectra of the k species of an unknown mixture, according to the equation D=CS^{T}+E, wherein E is an error matrix containing the residuals of the data (Ruckebusch & Blanchet, Analytica Chimica Acta 765, 2013, 28-36). The MCR method has been adapted to decompose and analyse ToF-SIMS. Commercially available software can be used for this task, e.g. the software package SurfaceLab Ver 7.1., wherein optionally the number of factors is set to 3, 4 or 5. A general account of how spectral information can be dissected is given by Juan & Tauler (Analytica Chimica Acta 1145, 2021, 59-78).

Summing up, the ToF-SIMS result measured in a specific coating or container can be attributed a specific position in an n-dimensional compositional space. MCR is used to reduce the complexity of the ToF-SIMS result by summarizing the datasets into a more limited number of variables, the so-called “factors”. The results of the MCR are a set of factors, loadings and corresponding scores. Each of the factors has a factor-specific MCR loading, indicating a conceptional component in said n-dimensional compositional space which can be attributed to said factor, wherein the loading characterizes the factor in that it lists the ions that contribute to the definition of said factor. Each factor relates to substances present in or on the coating or container. To be clear, the conceptional component is not in fact present in the coating or container. Each score indicates the intensity of the corresponding factor. It correlates with the abundance of substances in or on the coating or container.

### Reference Lipid Nanoparticles (LNP)

The coated glass vial was treated with either a phosphate-buffered saline (PBS) solution or a Reference-LNP in the same PBS solution. The differently treated coated glass vial were subjected to the above described ToF-SIMS measurement and data were extracted according to the above described MCR analysis.

In a first variant, the reference LNP-composition was the Comirnaty vaccine (license number EU/1/20/1528).

In a second variant, the reference-LNP contained the following lipids in the indicated amounts: 7.2 mg/mL (4-hydroxybutyl)azanediyl)bis(hexane-6,1-diyl)bis(2-hexyldecanoate), 0.83 mg/mL 2 [(polyethylene glycol)-2000]-N,N-ditetradecylacetamide, 1.5 mg/mL 1,2-distearoyl-sn-glycero-3-phosphocholine, and 3.3 mg/mL cholesterol, in phosphate-buffered saline (PBS) (pH 7.4) with a saccharide content of 10 wt. % and the following concentrations

| Compound | Concentration | Concentration |
|---|---|---|
| (salt or sugar) | (mmol/L) | (g/L) |
| NaCl | 137 | 8.0 |
| KCl | 2.7 | 0.2 |
| Na₂HPO₄ | 10 | 1.42 |
| KH₂PO₄ | 1.8 | 0.24 |
| Sucrose | | 100 |

### Further LNP Formulations

LNPs with similar formulations may alternatively be used which formulations may deviate up to 30 wt. % from the given quantities of individual lipid components.

Additionally the LNPs contain RNA, such as mRNA, particularly based on polynucleotides containing adenine.

### Abstract

A method for evaluating suitability of a pharmaceutical container for lipid-based carrier systems. Lipid-based carrier systems are sensitive pharmaceutical vehicles which require a pharmaceutical container that meets the necessary standards for storage and shipment.

What is claimed is:

1-35. (canceled)

36. A method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface;
selecting between 10 to 1000 signals from the ToF-SIMS data;
performing MCR (Multivariate Curve Resolution) analysis on the selected signals from the ToF-SIMS data, obtaining one or more factors;
obtaining corresponding scores for the one or more factors from the MCR analysis; and
evaluating at least one score of the one or more factors by comparison to a reference score.

37. The method as recited in claim 36 wherein the pharmaceutical container is a syringe, a cartridge, an ampoule or a vial, wherein the pharmaceutical container is a glass container or a polymer container.

38. The method as recited in claim 36 wherein the step incubating the container with a lipid-based carrier system comprises the following steps:
filling the container with a reference composition, containing a lipid-based carrier system,
storing the filled container,
emptying, cleaning and drying the container.

39. The method as recited in claim 38 wherein
the filled container is stored for a period of at least 3 hours, or at least 6 hours;
the filled container is stored at a temperature below 10° C. or
the filled container is stored at a temperature below −10° C., or even below −50° C.

40. The method as recited in claim 38 wherein before filling the container with the reference composition, the container is cleaned.

41. The method as recited in claim 38 wherein the reference composition
comprises a lipid-based carrier system, such as LNPs, and water, and optionally a buffer; or
comprises or is the Comirnaty® vaccine drug product (license number EU/1/20/1528).

42. The method as recited in claim 36 wherein the one or more factors include lipid-factor1, silicon-organic factor1, silicon-inorganic factor1 or organic factor1.

43. The method as recited in claim 36 wherein in the step of performing MCR analysis on the ToF-SIMS data, each of the one or more factors has a factor-specific MCR loading indicating a component in a n-dimensional compositional space attributable to each of the one or more factors, wherein the factor-specific MCR loading characterizes the one or more factors by listing ions that contribute to the definition of the factor.

44. The method as recited in claim 42 wherein
the lipid factor1 includes, in its factor specific MCR loading, one or more of the following ions: fatty acid-ions; [CₙH₂ₙ₋₁O₂]⁻, wherein n is 10, 12, 14, 16 or 18; [CₙH₂ₙ₋₃O₂]⁻, wherein n is 10, 12, 14, 16 or 18; [CₙH₂ₙ₋₅O₂]⁻, wherein n is 16 or 18; phosphatidyl-choline ions; [(CH)ₙH₂O₄P]⁻, wherein n=0, 1, 2 or 3, or
the silicon-organic factor1 includes, in its factor specific MCR loading, one or more of the following ions: silane species, silicon-carbon species, polysiloxane species based on the formula [OSiR₁R₂]ₙ⁻, wherein R₁ and R₂ are independently selected from methyl, ethyl, propyl, and wherein n is any integer between 2 and 10; or
the silicon-inorganic factor1 includes, in its factor specific MCR loading, one or more of the following ions: silicon species; aluminium-species and/or boron species; halogen species.

45. The method as recited in claim 42 wherein
lipid factor1 includes, in its factor specific MCR loading, one or more of the following ions: [C₁₀H₁₇O₂]⁻, [C₁₀H₁₉O₂]⁻, [C₁₂H₂₁O₂]⁻, [C₁₆H₂₉O₂]⁻, [C₁₆H₃₁O₂]⁻, [C₁₆H₃₂O₂]⁻, [C₁₈H₃₁O₂]⁻, [C₁₈H₃₃O₂]⁻, [C₁₈H₃₅O₂]⁻, [PO₃], [PH₂O₄]⁻, [CH₃O₄P]⁻, [C₂H₄O₄P]⁻;
silicon-organic factor1 includes, in its factor specific MCR loading, one or more of the following ions: [SiC]⁻, [SiCH₃O]⁻, [SiCH₃O₂]⁻, [SiC₂H₅O]⁻, [Si₂CHO₂]⁻, [SiC₃H₉O]⁻, [Si₂C₅H₁₅O₂]⁻, [Si₃C₅H₁₅O₄]⁻; and/or
silicon-inorganic factor1 includes, in its factor specific MCR loading, one or more of the following ions: OH⁻, Al⁻, Si⁻, P⁻, Cl⁻, NaO⁻, AlO⁻, BO₂⁻, SiHO⁻, AlO₂⁻, SiO₂⁻, SiH₅O₂⁻, Si₃H₃O₂, Si₂HO₅⁻.

46. The method as recited in claim 42 wherein
lipid factor1 includes, in its factor specific MCR loading, at least the following ions: [C₁₀H₁₉O₂]⁻, [C₁₂H₂₁O₂]⁻, [C₁₆H₂₉O₂]⁻, [C₁₆H₃₁O₂]⁻, and [C₁₈H₃₅O₂]⁻;
silicon-organic factor1 includes, in its factor specific MCR loading, at least the following ions: [SiCH₃O]⁻, [SiCH₃O₂]⁻, [SiC₂H₅O]⁻, [SiC₃H₉O]⁻, and [Si₂C₅H₁₅O₂]⁻; and/or
silicon-inorganic factor1 includes, in its factor specific MCR loading, at least the following ions: OH⁻, Si⁻, SiO₂, SiH₅O₂⁻, and Si₃H₃O₂⁻.

47. The method as recited in claim 36 wherein the step evaluating the score of the one or more factors by comparison to a reference score comprises one or more of the following steps
obtaining the reference score from a reference container; or
obtaining a reference value for the reference score.

48. The method as recited in claim 36 wherein the step evaluating the score of the one or more factors by comparison to a reference score comprises one or more of the following steps:
comparing the score of the pharmaceutical container with the reference score;
assessing whether a difference of the score of the pharmaceutical container and the reference score is below or above a pre-set threshold value; or
assessing whether a quotient of the score of the pharmaceutical container and the reference score is below or above a pre-set threshold value.

49. The method as recited in claim 36 wherein the evaluation results in a positive or negative answer indicating fitness of suitability or lack of suitability respectively.

50. The method as recited in claim 36 wherein the comparison to a reference score comprises the following steps:
providing a reference container having an inner surface and an outer surface, wherein optionally at least part of the inner surface is coated with a coating, wherein the reference container has the same dimensions and the same composition as the pharmaceutical container;
incubating the reference container with a lipid-based carrier system;
acquiring ToF-SIMS data from the inner surface of the reference container;
selecting between 10 to 1000 signals from the ToF-SIMS data;
performing MCR (Multivariate Curve Resolution) analysis on the ToF-SIMS data obtaining one or more factors;
obtaining corresponding scores for the one or more factors from the MCR analysis;
assessing whether the difference of the score of the one or more factors of the pharmaceutical container and the score of the one or more factors of the reference pharmaceutical container is below or above a pre-set threshold value, or assessing whether the quotient of the score of the one or more factors of the pharmaceutical container and the score of the one or more factors of the reference pharmaceutical container is below or above a pre-set threshold value.

51. A method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the ToF-SIMS data obtaining one or more factors;
selecting one or more factors, wherein the one or more factors include lipid-factor1;
obtaining a corresponding score for lipid-factor1 from the MCR analysis;
dividing the score of lipid-factor1 by the analogous score of a reference pharmaceutical container to obtain a quotient for lipid-factor1; and
assessing suitability of the pharmaceutical container for lipid-based carrier systems based on the criterion whether the quotient for lipid-factor1 is less than 0.5.

52. A method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the ToF-SIMS data obtaining one or more factors;
selecting one or more factors, wherein the one or more factors include silicon-organic factor1;
obtaining a corresponding score for silicon-organic factor1 from the MCR analysis;
dividing the score of silicon-organic factor1 by the analogous score of a reference pharmaceutical container to obtain a quotient for silicon-organic factor1; and
assessing suitability of the pharmaceutical container for lipid-based carrier systems based on the criterion whether the quotient for silicon-organic factor1 is more than 2.0.

53. A method for evaluating suitability of a pharmaceutical container for lipid-based carrier systems comprising the following steps:
providing a pharmaceutical container having an inner surface and an outer surface, wherein at least part of the inner surface is coated with a coating;
incubating the container with a lipid-based carrier system;
acquiring ToF-SIMS data from the inner surface of the container;
selecting between 10 to 1000 signals from the ToF-SIMS data;
performing MCR (Multivariate Curve Resolution) analysis on the ToF-SIMS data obtaining one or more factors;
selecting one or more factors, wherein the one or more factors include organic factor1;
obtaining a corresponding score for organic factor1 from the MCR analysis;
dividing the score of organic factor1 by the analogous score of a reference pharmaceutical container to obtain a quotient for organic factor1; and
assessing suitability of the pharmaceutical container for lipid-based carrier systems based on the criterion whether the quotient for organic factor1 is more than 0.2.

54. The method as recited in claim 36 wherein the pharmaceutical container has one or more of the following properties:
a wall thickness between 0.50 and 10.0 mm, or
a volume capacity of the container of 0.1 ml to 1000 ml.

55. The method as recited in claim 36 wherein the pharmaceutical container has one or more of the following properties:
a glass composition comprising 50 to 90 wt. % SiO₂, and 3 to 25 wt. % B₂O₃, or
a glass composition comprising aluminosilicate.

56. The method as recited in claim 36 wherein the pharmaceutical container has a glass composition comprising 70 to 81 wt. % SiO₂, 1 to 10 wt. % Al₂O₃, 6 to 14 wt. B₂O₃, 3 to 10 wt. % Na₂O, 0 to 3 wt. % K₂O, 0 to 1 wt. % Li₂O, 0 to 3 wt. % MgO, 0 to 3 wt. % CaO, 0 to 5 wt. % BaO.

57. The method as recited in claim 36 wherein the pharmaceutical container has a glass composition comprising 72 to 82 wt. % SiO₂, 5 to 8 wt. % Al₂O₃, 3 to 6 wt. B₂O₃, 2 to 6 wt. % Na₂O, 3 to 9 wt. % K₂O, 0 to 1 wt. % Li₂O, 0 to 1 wt. % MgO, 0 to 1 wt. % CaO.

58. The method as recited in claim 36 wherein the pharmaceutical container has a glass composition comprising 60 to 78 wt. % SiO₂, 7 to 15 wt. B₂O₃, 0 to 4 wt. % Na₂O, 3 to 12 wt. % K₂O, 0 to 2 wt. % Li₂O, 0 to 2 wt. % MgO, 0 to 2 wt. % CaO, 0 to 3 wt. % BaO, 4 to 9 wt. % ZrO₂.

59. The method as recited in claim 36 wherein the pharmaceutical container has a glass composition comprising 50 to 70 wt. % SiO₂, 10 to 26 wt. % Al₂O₃, 1 to 14 wt. B₂O₃, 0 to 15 wt. % MgO, 2 to 12 wt. % CaO, 0 to 10 wt. % BaO, 0 to 2 wt. % SrO, 0 to 8 wt. % ZnO, 0 to 2 wt. % ZrO₂.

60. The method as recited in claim 36 wherein the pharmaceutical container has a glass composition comprising 55 to 70 wt. % SiO₂, 11 to 25 wt. % Al₂O₃, 0 to 10 wt. % MgO, 1 to 20 wt. % CaO, 0 to 10 wt. % BaO, 0 to 8.5 wt. % SrO, 0 to 5 wt. % ZnO, 0 to 5 wt. % ZrO₂, 0 to 5 wt. % TiO₂.

61. The method as recited in claim 36 wherein the pharmaceutical container has a glass composition comprising 65 to 72 wt. % SiO₂, 11 to 17 wt. % Al₂O₃, 0.1 to 8 wt. % Na₂O, 0 to 8 wt. % K₂O, 3 to 8 wt. % MgO, 4 to 12 wt. % CaO, 0 to 10 wt. % ZnO.

62. The method as recited in claim 36 wherein the pharmaceutical container has a glass composition comprising 64 to 78 wt. % SiO₂, 4 to 14 wt. % Al₂O₃, 0 to 4 wt. % B₂O₃, 6 to 14 wt. % Na₂O, 0 to 3 wt. % K₂O, 0 to 10 wt. % MgO, 0 to 15 wt. % CaO, 0 to 2 wt. % ZrO₂, 0 to 2 wt. % TiO₂.

63. A method comprising the following steps:
producing a first and a second coated container with a same production method;
evaluating the first coated container by the method as recited in claim 36, to obtain results of the evaluation of the first coated container; and
applying the results of the evaluation of the first coated container to determine the suitability of the second coated container for the storage of pharmaceutical composition, or using the results of the evaluation for the quality control of the first or second container.

64. A pharmaceutical container evaluated according to the method as recited in claim 36 comprising the lipid-based carrier system.

65. A pharmaceutical container comprising information attached comprising the results of the evaluation of claim 36.

66. A kit, comprising:
i) a coated container and
ii) a data sheet or storage medium comprising the results of the evaluation of claim 36.
